(19) United States
(12) Patent Application Publication
(10) Pub. No.: US 2016/0245809 A1
(43) Pub. Date: Aug. 25, 2016

(54) Ligand Conjugated Quantum Dots for the Detection of Soluble Receptors and Exosomes in Biological Fluids

(71) Applicant: The Penn State Research Foundation, University Park, PA (US)

(72) Inventors: James Robert Connor, Hershey, PA (US); Achuthamangalam B. Madhankumar, Hummelstown, PA (US)

(21) Appl. No.: 15/048,180

(22) Filed: Feb. 19, 2016

Related U.S. Application Data
(60) Provisional application No. 62/118,701, filed on Feb. 20, 2015.

(51) Int. Cl. G01N 33/566

(52) U.S. Cl. CPC G01N 33/566; G01N 2333/7155; G01N 2800/7028

(57) ABSTRACT

The present invention relates to compositions and method for diagnosing cancer by detecting IL13Rα2 or a tumor associated exosome. In some instances, the invention relates to using IL13-conjugated quantum dots to detect an IL13Rα2 or a tumor associated exosome.

### CROSS-REFERENCE TO RELATED APPLICATIONS

This application claims priority to U.S. Provisional Patent Application No. 62/118,701 filed Feb. 20, 2015, the contents of which are incorporated by reference herein in their entirety.

### BACKGROUND OF THE INVENTION

The cerebrospinal fluid (CSF) and serum of the brain tumor patients often exhibit an abnormal vesicle and its associated membrane protein expression pattern (Liu et al., 2014, Int J Clin Exp Pathol 7:4857-66). Tumor cell secretome and extracellular vesicles (exosomes) based biomarkers in the biofluid are being explored recently in the neuro-oncology research for staging and to determine the therapeutic response (Shao et al., 2012, Nat Med 18:1835-40; Formolo et al., 2011, J Proteome Res 10:3149-59). The nature and composition of proteins in the exosomes may provide the earliest indication of disease relapse or progression in the central nervous system. Exosomes plays a significant role in the cancer progression and invasion in the CNS malignancies, by altering the tumor microenvironment (Liu et al., 2014, Int J Clin Exp Pathol 7:4857-66; Bronisz et al., 2014, Cancer Res 74:738-50; D'Asti et al., 2012, Front Physiol 3:294). The presence of exosomes in the CSF samples may provide evidence for the presence of glioma tumor cells or even glioma stem cells in the brain.

Quantum dots are cadmium and selenium based semiconductor nanoparticles ranging in size from 1-20 nm. These particles have a variety of applications in physiological systems from drug delivery to disease diagnosis (Fatehi et al., 2014, J Nanosci Nanotechnol 14:5355-62; Mahajan et al., 2012, Methods Enzymol 509:41-60; Feng et al., 2013, Cell Mol Neurobiol 33:759-65). Tumor cells secrete a variety of soluble factors and extracellular vesicles (exosomes) in the serum and cerebrospinal fluid very early in the course of tumor recurrence, progression and as a prelude to metastasizing. For example, IL13Rα2 receptor is one type of oncogenic receptor expressed in several malignancies including glioblastoma (GBM), ovarian cancer, renal cell carcinoma and even in recurrent glioma (Husain et al., 2001, Int J Cancer 92:168-75). IL13Rα2 receptor expression is a marker of tumor malignancy and invasiveness (Debinski et al., 2000, J Neurooncol 48:103-11; Obiri et al., 1995, J Biol Chem 270:8797-804; Murata et al., 1997, Int J Cancer 70:230-40). IL13 has specific binding affinity to IL13Rα2, which is overexpressed in cancer cells compared to its normal physiological receptor, IL13Rα1. In a previous study it was demonstrated that the therapeutic potential of this marker in the form of IL13 linked nanovesicles (Madhankumar et al., 2006, Mol Cancer Ther 5:3162-9; Madhankumar et al., 2009, Mol Cancer Ther 8:648-54). It would be worthwhile to study the interaction of IL13QD with the glioma stem cells and exosomes to investigate its suitability as a detection tool.

A tumor marker that allows simple, reliable, early detection of new or recurrent cancer from easily accessible biologic fluids is the goal of clinical oncologists and public health departments worldwide. Sadly, very few such cancer associated markers in blood, urine or cerebrospinal fluid have been discovered, and none exist for malignant GBM.

Thus, there a need in the art for compositions and methods for detecting malignant GBM. The present invention addresses this unmet need in the art.

### SUMMARY OF THE INVENTION

In one aspect, the invention provides a method of detecting an IL13-specific receptor in a sample. In one embodiment, the method comprises contacting a portion of the sample with a IL13 conjugated quantum dot (IL13QD) under conditions suitable for binding of the IL13QD dot with an IL13-specific receptor and detecting the IL13-specific receptor.

In one embodiment, the detecting step comprises one or more of fluorescence microscopy, flow cytometry, immunoassay, atomic force microscopy, and agarose gel electrophoresis.

In one embodiment, the quantum dot comprises a material selected from the group consisting of cadmium selenide, zinc sulfur, silicon and any combination thereof. In another embodiment, the quantum dot is surface modified with at least one selected from the group consisting of polyethylene glycol (PEG) and a carboxylic acid functional group.

In one embodiment, the sample comprises one or more of serum and cerebrospinal fluid.

In one embodiment, the IL13-specific receptor is IL13Rα2.

In another aspect, the invention provides a method of detecting an exosome in a sample. In one embodiment, the method comprises contacting a portion of the sample with a IL13 conjugated quantum dot under conditions suitable for binding of the IL13 conjugated quantum dot with an exosome and detecting the exosome.

In one embodiment, the exosome is a tumor associated exosome. In another embodiment, the exosome expresses an IL13-specific receptor. In yet another embodiment, the IL13-specific receptor is IL13Rα2.

In one embodiment, the quantum dot is surface modified with at least one selected from the group consisting of polyethylene glycol (PEG) and a carboxylic acid functional group.

The present invention also provides a method for diagnosing cancer in a subject. In one embodiment, the method comprises determining the level of IL13 conjugated quantum dot (IL13QD) binding to a target; comparing the level of the IL13QD binding to the target in the biological sample of the subject with a comparator control; and diagnosing the subject with cancer when the level of the IL binding to the biological sample of the subject is altered at a statistically significant amount when compared with the level of IL13QD binding to the comparator control.

In some embodiments, the step determining the level of IL13QD biding to a target further comprises obtaining a biological sample of the subject; contacting a portion of the biological sample with a IL13QD; and determining the level of IL13QD binding to the target in the biological sample.

In another embodiment, the step determining the level of IL13QD biding to a target further comprises administering to the subject a composition comprising the IL13QD; and determining the level of IL binding to the target in situ.

In one embodiment, the biological sample is selected from the group consisting of serum, cerebrospinal fluid, urine and an exosome isolated from cerebrospinal fluid.

In one embodiment, the determining the level of IL13QD binding step comprises one or more of fluorescence microscopy, flow cytometry, immunoassay, atomic force microscopy, and agarose gel electrophoresis.

In one embodiment, the level of the IL13QD binding to the biological sample of the subject is increased at a statistically significant amount when compared with the level of IL13QD binding to the comparator control.

In one embodiment, the quantum dot comprises a material selected from the group consisting of cadmium selenide, zinc sulfur, and any combination thereof.

In one embodiment, the cancer is selected from the group consisting of adenocarcinoma, pancreatic cancers sarcoma, peripheral nerve tumors, and glioblastoma.

The invention also provides a conjugated quantum dot, wherein the conjugated quantum dot binds to an IL13-specific receptor. In one embodiment, the conjugated quantum dot comprises IL13.

In another aspect, the invention provides a kit for detecting an IL13-specific receptor, the kit comprising an IL13 conjugated quantum dot.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings

FIG. 1, comprising FIG. 1A and FIG. 1B, depicts results from experiments demonstrating that IL13 conjugated effectively on the quantum dots which bears carboxyl groups on the surface of CdSe/ZnS core shell. FIG. 1A depicts an image showing that pegylated quantum dots were conjugated to recombinant IL13 protein by EDC chemistry by utilizing the carboxylic acid groups in the quantum dot and the amino groups on the lysine residues in the IL13 protein. FIG. 1B depicts an agarose gel demonstrating the conjugation of IL13 to the quantum dots was confirmed by agarose gel electrophoresis, where the conjugated quantum dots had retardation in their electrophoretic mobility compared to the unconjugated quantum dots.

FIG. 2, comprising FIG. 2A through FIG. 2C, depicts results of experiments demonstrating that the size of the quantum dots were uniformly distributed with approximate size around 15-20 nm. FIG. 2A depicts the surface modified quantum dots have uniform particle size distribution, with an average particle size of 12.8 nm for non-targeted quantum dots and 24.1 nm for the IL13 conjugated quantum dots. FIG. 2B depicts atomic force microscopy data confirming the spherical and surface morphology and the particle size of the IL13QD. FIG. 2C depicts transmission electron microscopy data confirming the spherical and surface morphology and the particle size of the IL13QD.

FIG. 3 depicts immunocytochemistry data indicating that T3691 glioma stem cells in the monolayer and in the aggregated spindle and sphere form express IL13Rα2 receptor. DAPI stains the nucleus.

FIG. 4, comprising FIG. 4A and FIG. 4B, depicts results of experiments demonstrating the binding of IL13 QD. FIG. 4A depicts data showing IL13 QD selectively binds the glioma stem cells both in the monolayer (top panels) and in the spheroid form (bottom panels). FIG. 4B depicts data showing unconjugated quantum dots do not bind the glioma stem cells. Similarly binding of the Il13QD to the stem cells does not occur if the cells are pretreated with 100 fold excess of IL13.

FIG. 5, comprising FIG. 5A and FIG. 5B, depicts results of experiments demonstrating IL13QD is able to bind effectively and selectively to the T3691 glioma stem cells. FIG. 5A depicts IL13Rα2 expression in T387 glioma stem cells. FIG. 5B depicts data demonstrating that unconjugated quantum dots (UNC-QD) do not bind the glioma stem cells (upper panel). Similarly binding of the IL13QD to the stem cells did not occur when the cells were pretreated with 100 fold excess of IL-13 (bottom panel).

FIG. 6, comprising FIG. 6A through FIG. 6D, depicts results of experiments demonstrating the force of interaction between the IL13QD and the glioma cells and exosomes. FIG. 6A depicts force curve generated after interaction between the non-targeted QD's fixed on the atomic force microscopy probe (AFM) and the U251 glioma cells before exposure of the probe and the cell (t=1 second). FIG. 6B depicts force curve generated after interaction between the non-targeted QD's fixed on the atomic force microscopy probe (AFM) and the U251 glioma cells before after of the probe and the cell (t=1 second). A smooth curve demonstrates negligible force of interaction between the AFM probe and the receptors on the cells. It is also clear that there is a time dependent increase in the force of interaction between the IL13QD and the receptors on the cells. FIG. 6C depicts the magnitude of binding force between the IL13QD and the U251 glioma cells. IL13QD-0 and IL13QD-1 indicates the binding force between the AFM probe and the glioma cells at no contact delay (t=0 sec) and contact delay of 1 second respectively, similarly represents the binding force between the probe and the unconjugated QD. FIG. 6D depicts the binding force between the AFM probe linked with quantum dots and exosomes indicates a higher binding force between the IL13QD and T3691 exosomes coated on coverslips compared to control (BSA-QD). To minimize the non-specific interaction between the quantum dots and biobond coated coverslip, control coverslips were coated with BSA. Error bars represents standard deviation.

FIG. 7, comprising FIG. 7A through FIG. 7D, depicts transmission electron microscopic images of the IL13QD and the exosomes secreted by glioma stem cells after staining with uranyl acetate. FIG. 7A depicts T387 exosomes before exposing to IL13QD. FIG. 7B depicts T387 exosomes after exposure to IL13QD. Arrows indicates the binding of the IL13QD on the exosomes isolated from T387 glioma stem cells. FIG. 7C depicts binding of IL13QD to the exosomes isolated from the CSF of glioma patient P745. FIG. 7D depicts a representation the interaction of unconjugated quantum dots and T387 exosomes.

FIG. 8 depicts forward and sideward scattering (FSC and SSC respectively) from the flow cytometry of the exosomes-QD complex indicating that there is a difference in the binding profile of the IL13 conjugated and non-conjugated quantum dots. This evidences the binding phenomenon of quantum dots to the exosomes to be receptor mediated.

FIG. 9 depicts flow cytometry performed on T3691-green fluorescence labelled exosomes after interaction with IL13 conjugated and BSA conjugated quantum dots. The forwards and reverse scattered particle populations are represented in the left panel. P6 represents the green fluorescence sorted particles.

FIG. 10, comprising FIG. 10A and FIG. 10B, depicts results from experiments demonstrating acetylcholine esterase, IL13Rα2 and CD63 expression of glioma stem cell exosomes. FIG. 10A depicts acetylcholine esterase activity of the exosomes. increases with increasing time interval from 10 minutes to 60 minutes. AChE activity is a general method of quantifying the exosomes. All the exosomes were isolated from the CSF of brain tumor patients (P620, P626, P636, P646) and T3691 glioma stem cells confirms the presence of exosomes. FIG. 10B depicts the expression of IL13Rα2 in the exosomes isolated from glioma stem cells (T3691 and T387). Expression of CD63 is also indicated as a loading control for the exosomes.

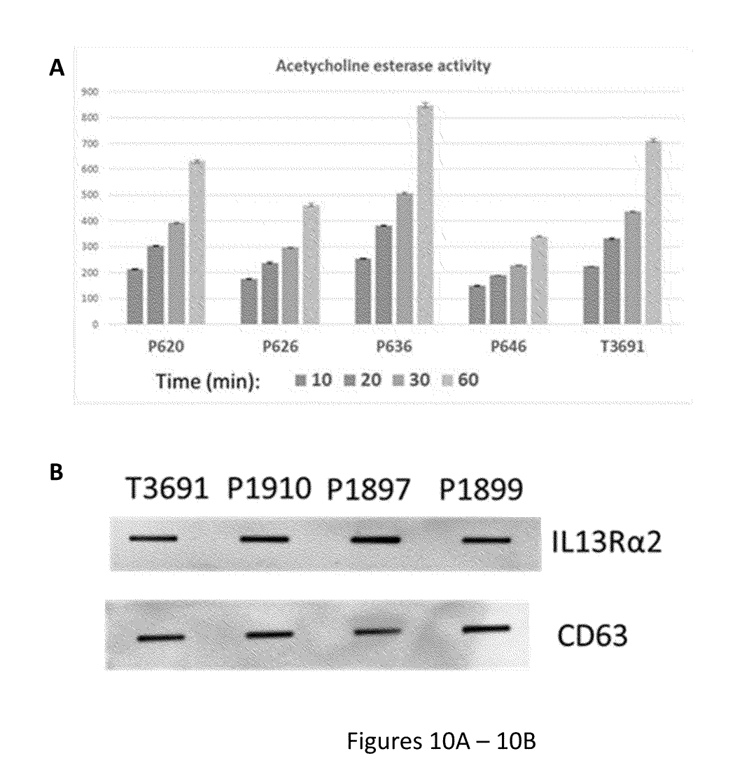

### DETAILED DESCRIPTION

The invention relates to the discovery that IL13 conjugated quantum dots (IL13QD), when exposed to soluble receptor or tumor associated exosomes, aggregate or dissociate depending on the molecules linked on the quantum dots and such aggregation and dissociation can be monitored by methods such as microscopy, flow cytometry or agarose gel electrophoresis. The results presented herein demonstrate the aggregation properties of the IL13QD in the presence of a receptor and exosomes, thereby indicating a correlation between the receptor expression and aggregation and dissociation of quantum dots. The present invention therefore provides methods and compositions for detecting an IL13-specific receptor.

Accordingly, in some embodiments of the invention, a method of detecting an IL13-specific receptor in a sample is provided. The method comprises contacting a portion of the sample with an IL13QD under conditions suitable for binding of the IL13QD with an IL13-specific receptor and detecting the IL13-specific receptor.

In other embodiments of the invention, a method of detecting tumor associated exosomes in a sample is provided. The method comprises contacting a portion of the sample with an IL13QD under conditions suitable for binding of the IL13QD with an exosome and detecting the exosome. In one embodiment, the exosome expresses an IL13-specific receptor. In some embodiments, the IL13-specific receptor is IL13Rα2.

In one embodiment, detecting an exosome or IL13-specific receptor comprises fluorescence microscopy, flow cytometry, immunoassay, atomic force microscopy, or agarose gel electrophoresis.

In some embodiments, the quantum dot is a cadmium selenide quantum dot. In other embodiments, the quantum dot can be surface modified with polyethylene glycol (PEG), streptavidin, or a carboxylic acid functional groups or a combination thereof.

This approach can be used to identify cancer associated exosomes in biological fluids such as cerebrospinal fluid (CSF) or urine. In addition to detection of exosomes, certain antigen conjugated nanoparticles form agglomerates in the presence of soluble antibodies in a given media.

Accordingly, the invention includes methods of diagnosing or prognosing cancer in a subject. In one embodiment, the method can be performed ex vivo, using a biological sample of the subject, or in situ, by administering a composition of quantum dots to detect cancerous cells. Accordingly, the method of diagnosing or prognosing cancer comprises determining the level of IL13 conjugated quantum dot (IL13QD) binding to a target, comparing the level of the IL13QD binding to the target in the biological sample of the subject with a comparator control, and diagnosing the subject with cancer when the level of the IL13QD binding to the biological sample of the subject is altered at a statistically significant amount when compared with the level of IL13QD binding to the comparator control.

To carry out the method of diagnosing or prognosing cancer ex vivo, the step determining the level of IL13QD biding to a target further comprises obtaining a biological sample of the subject, contacting a portion of the biological sample with a IL13QD, and determining the level of IL binding to the target in the biological sample.

To carry out the method of diagnosing or prognosing cancer in situ, the step determining the level of IL13QD biding to a target further comprises administering to the subject a composition comprising the IL13QD, and determining the level of IL binding to the target in situ.

The method comprises obtaining a biological contacting a portion of the biological sample with an IL13QD, determining the level of IL13QD binding to a target in the biological sample, comparing the level of the IL13QD binding to the target in the biological sample of the subject with a comparator control, and diagnosing the subject with cancer when the level of the IL13QD binding to the biological sample of the subject is altered at a statistically significant amount when compared with the level of IL13QD binding to the comparator control.

In certain embodiments, the subject is diagnosed with cancer when level of the IL binding to the biological sample of the subject is increased at a statistically significant amount when compared with the level of IL13QD binding to the comparator control.

In various embodiments, the invention provides receptor targeted quantum dots. In some embodiments, the receptor target is an IL13-specific receptor. In one embodiment, the receptor target is IL13Rα2.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate.

The term “quantum dot” (QD) refers to a semiconductor nanostructure that confines the motion of conduction band electrons, valence band holes, or excitons (bound pairs of conduction band electrons and valence band holes) in all three spatial directions. The confinement can be due to electrostatic potentials (generated by external electrodes, doping, strain, impurities), the presence of an interface between different semiconductor materials (e.g. in core-shell nanocrystal systems), the presence of the semiconductor surface (e.g. semiconductor nanocrystal), or a combination of these. A quantum dot has a discrete quantized energy spectrum. The corresponding wave functions are spatially localized within the quantum dot, but extend over many periods of the crystal lattice. A quantum dot contains a small finite number (of the order of 1-100) of conduction band electrons, valence band holes, or excitons, i.e., a finite number of elementary electric charges. One of the optical features of small excitonic quantum dots immediately noticeable to the unaided eye is coloration. While the material which makes up a quantum dot defines its intrinsic energy signature, more significant in terms of coloration is the size. The larger the dot, the redder (the more towards the red end of the spectrum) the fluorescence. The smaller the dot, the bluer (the more towards the blue end) it is. The coloration is directly related to the energy levels of the quantum dot. Quantitatively speaking, the bandgap energy that determines the energy (and hence color) of the fluoresced light is inversely proportional to the square of the size of the quantum dot.

The term “conjugate” refers to a physical or chemical attachment of one molecule to a second molecule.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “marker” or “biomarker” is meant to include a parameter which is useful according to this invention for determining the presence and/or severity of cancer.

The level of a marker, biomarker, or quantum dot binding “significantly” differs from the level of the marker, biomarker, or quantum dot binding in a reference sample if the level in a sample from the patient differs from the level in a sample from the reference subject by an amount greater than the standard error of the assay employed to assess the marker, and preferably at least 10%, and more preferably 25%, 50%, 75%, or 100%.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

“Differentially increased binding” refers to quantum dot binding levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments there between than a control.

“Differentially decreased binding” refers to refers to quantum dot binding levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments there between than a control.

The “level” of one or more biomarkers means the absolute or relative amount or concentration of the biomarker in the sample.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “providing a prognosis” refers to providing a prediction of the probable course and disease or disorder, including prediction of severity, etc. The methods can also be used to devise a suitable therapeutic plan.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype.

“Sample” or “biological sample” as used herein means a biological material isolated from an individual, including but is not limited to organ, tissue, exosome, blood, plasma, saliva, urine and other body fluid. The biological sample may contain any biological material suitable for detecting the desired biomarkers, and may comprise cellular and/or non-cellular material obtained from the individual.

The term “cancer” as used herein is defined as disease characterized by the abnormal growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, sarcoma and the like.

“Standard control value” as used herein refers to a predetermined amount of a particular protein or nucleic acid that is detectable in a biological sample, for example, blood, either in whole blood or in blood supernatant. The standard control value is suitable for the use of a method of the present invention, in order for comparing the amount of a protein or nucleic acid of interest that is present in a biological sample. An established sample serving as a standard control provides an average amount of the protein or nucleic acid of interest in the biological that is typical for an average, healthy person of reasonably matched background, e.g., gender, age, ethnicity, and medical history. A standard control value may vary depending on the protein or nucleic acid of interest and the nature of the sample (e.g., whole blood or supernatant).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based in part, on the discovery that IL13-conjugated quantum dots (IL13QD) can detect IL13Rα2 in cerebrospinal fluid of glioblastoma muliforme (GBM) patients or tumor associated exosomes.

In one aspect, the invention provides a method for detecting an IL13-specific receptor or a tumor associated exosome. In one embodiment, the method comprises contacting a portion of the sample with a IL13QD under conditions suitable for binding of the IL13QD with an exosome or an IL13-specific receptor and detecting the exosome or the or an IL13-specific receptor.

IL13Rα2 receptor expressing exosomes plays a significant role in the tumorigenic property. Thus, in one aspect, the invention provides a method for diagnosing or prognosing cancer in a subject. In one embodiment the method comprises determining the level of IL13 conjugated quantum dot (IL13QD) binding to a target, comparing the level of the IL13QD binding to the target in the biological sample of the subject with a comparator control, and diagnosing the subject with cancer when the level of the IL13QD binding to the biological sample of the subject is altered at a statistically significant amount when compared with the level of IL13QD binding to the comparator control. In one embodiment, determining the level of IL13QD biding to a target further comprises obtaining a biological sample of the subject, contacting a portion of the biological sample with a IL13QD, and determining the level of IL13QD binding to the target in the biological sample. In another embodiment, determining the level of IL13QD biding to a target further comprises administering to the subject a composition comprising the IL and determining the level of IL binding to the target in situ.

### Methods of Diagnosis and Prognosis

The present invention has application in various diagnostic assays, including, but not limited to, the detection of cancer. Accordingly, the present invention features methods for diagnosing and prognosing cancers including detection of early tumor recurrence, and to distinguish recurrent tumor from pseudoprogression.

The invention provides for improved diagnosis of cancers. The cancer can be diagnosed or prognosed by measuring the binding of the IL13QD described herein to a biological sample and comparing the measured values to reference or index values. Such a comparison can be undertaken with mathematical algorithms or formula in order to combine information from results of multiple individual biomarkers and other parameters into a single measurement or index.

The IL13QD binding of the present invention can thus be used to generate a profile of subjects: (i) who do not cancer and/or (ii) who have cancer. The level of IL13QD binding of the present invention can prognose a patient or correlate with the disease pathology. The level of IL13QD binding in a biological sample of the subject can be compared to a predetermined or reference biomarker profile to diagnose or identify subjects having cancer, as well as the rate of progression of cancer, and to monitor the effectiveness of cancer related condition treatments.

In one embodiment, the method comprises determining the level of IL13 conjugated quantum dot (IL13QD) binding to a target, comparing the level of the IL13QD binding to the target in the biological sample of the subject with a comparator control, and diagnosing the subject with cancer when the level of the IL13QD binding to the biological sample of the subject is altered at a statistically significant amount when compared with the level of IL13QD binding to the comparator control. In one embodiment, determining the level of IL13QD biding to a target further comprises obtaining a biological sample of the subject, contacting a portion of the biological sample with a IL13QD, and determining the level of IL binding to the target in the biological sample. In another embodiment, determining the level of IL13QD biding to a target further comprises administering to the subject a composition comprising the IL and determining the level of IL binding to the target in situ.

In one embodiment, the method comprises detecting IL13QD binding in a biological sample of the subject. Preferably, the biological sample is CSF. In various embodiments, the level IL13QD binding in the biological sample of the subject is compared with the level of a corresponding IL13QD binding in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

In various embodiments, the subject is a human subject, and may be of any race, sex and age.

Information obtained from the methods of the invention described herein can be used alone, or in combination with other information (e.g., disease status, disease history, vital signs, blood chemistry, etc.) from the subject or from the biological sample obtained from the subject.

In other various embodiments of the methods of the invention, the level of IL13QD binding is determined to be increased when the level IL13QD binding is increased by at least 10%, by at least 20%, by at least 30%, by at least 40%, by at least 50%, by at least 60%, by at least 70%, by at least 80%, by at least 90%, or by at least 100%, when compared to with a comparator control.

In the methods of the invention, a biological sample from a subject is assessed for the level of IL13QD binding in the biological sample obtained from the patient. The level of IL13QD binding in the biological sample can be determined by microscopy, flow cytometry, agarose gel electrophoresis, or a combination thereof.

The following are non-limiting examples of cancers that can be diagnosed by the disclosed methods and compositions: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocvtoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

In some embodiments, the methods and compositions of the invention diagnose adenocarcinoma, pancreatic cancers GBM, ovarian cancer, renal cell carcinoma recurrent glioma, sarcoma, and peripheral nerve tumors.

### Quantum Dots

In one aspect, the invention provides a conjugated quantum dot. In one embodiment, the quantum comprises one or more materials.

The composition of the quantum dot is not particularly limited. Any core of the II-VI semiconductors (e.g., ZnS, ZnSe, ZnTe, CdS, CdSe, CdTe, HgS, HgSe, HgTe, and mixtures thereof), III-V semiconductors (e.g., GaAs, InGaAs, InP, InAs, and mixtures thereof) or IV (e.g., Ge, Si) semiconductors can be used in the context of the present disclosure. The core particle may have a core/shell structure, and each of the core and shell of the core particle may include the above-described compounds. The above-described compounds may be included in the core or shell alone or in combination of at least two thereof. For example, the core particle may have a CdSe—ZnS (core/shell) structure in which CdSe is contained as the core and ZnS is contained as the shell. In one embodiment, the quantum dot comprises CdSe, ZnS, or a combination thereof. The size of the quantum dot may be, but is not particularly limited to, for example 1 to 100 nm, preferably 1 to 50 nm.

In some embodiments the quantum dot is surface modified. In one embodiment, the quantum dot is surface modified with polyethylene glycol (PEG), carboxylic acid functional groups, primary amine functional groups, streptavadin or a combination thereof.

In one embodiment, the quantum dot is conjugated to IL13. In certain embodiments, the quantum dot is conjugated to IL13 by EDC chemistry using EDC as conjugating agent. In another embodiment, the conjugated quantum dot is purified using gel filtration.

The conjugated quantum dot of the invention may also comprise a linker that attaches the quantum dot to IL13 indirectly. The term “linker” denotes any chemical compound, which may be present between the quantum dot and IL13. This linker may be removed from the bioconjugated nanoparticle by chemical means, by enzymatic means, or spontaneously. The linker may be pharmacologically inert or may itself provide added beneficial pharmacological activity. The term “spacer” may also be used interchangeably as a synonym for linker. Linkers used in the present disclosure may include lipids, polypeptides, oligonucleotides, polymers, and the like. In some embodiments, a streptavidin conjugated quantum dot is linked to biotinylated IL13.

Quantum dots may be synthesized in various ways. Some common methods include (1) the spontaneous generation in quantum well structures due to monolayer fluctuations in the well's thickness; (2) the capability of self-assembled quantum dots to nucleate spontaneously under certain conditions during molecule beam epitaxy (MBE) and metallorganic vapor phase epitaxy (MOVPE), when the material is grown in a substrate to which it is not lattice matched; (3) the ability of individual quantum dots to be created from two-dimensional electron or hole gases present in remotely doped quantum wells or semiconductor heterostructures; and (4) chemical methods, such as synthesizing ZnTe quantum dots in high-temperature organic solution (see, e.g., Zhang, J. et al. Materials Research Society Symposium Proceedings, Vol. 942, 2006). These and other processes for the synthesis of QDs are well known in the art as disclosed, for example, by U.S. Pat. Nos. 5,906,670, 5,888,885, 5,229,320, 5,482,890, and Hines, M. A. J. Phys. Chem., 100, 468-471 (1996), Dabbousi, B. O. J. Phys. Chem. B, 101, 9463-9475 (1997), Peng, X., J. Am. Chem. Soc., 119, 7019-7029 (1997), which are incorporated herein by way of reference.

The quantum dots of the invention may be coated with a biodegradable or biocompatible material to render the quantum dots biocompatible when administered to a subject. As used herein, the term “biocompatible” relates to any synthetic or naturally occurring macromolecule, such as a lipid, carbohydrate, polysaccharide, protein, polymer, glycoproteins, glycolipids, etc., and methods of applying said biocompatible material, that can be used to coat the quantum dots to render it safe for in vivo use in a subject. Various methods of preparing lipid vesicles have been described including U.S. Pat. Nos. 4,235,871, 4,501,728, 4,837,028; PCT Application WO 96/14057, New RRC, Liposomes: A practical approach, IRL Press, Oxford (1990), pages 33-104; Lasic D D, Liposomes from physics to applications, Elsevier Science Publishers BV, Amsterdam, 1993; Szoka et al., Ann. Rev. Biophys. Bioeng. 9:467 (1980); Liposomes, Marc J. Ostro, ed., Marcel Dekker, Inc., New York, 1983, Chapter 1; Hope et al., Chem. Phys. Lip. 40:89 (1986); each of which is incorporated herein by reference.

Any lipid including surfactants and emulsifiers known in the art is suitable for use in making the inventive bioconjugated nanoparticles. The lipid component may also be a mixture of different lipid molecules. These lipids may be extracted and purified from a natural source or may be prepared synthetically in a laboratory. In a preferred embodiment, the lipids are commercially available. Lipids useful in coating the bioconjugated nanoparticles include natural as well as synthetic lipids. The lipids may be chemically or biologically altered. Lipids useful in preparing the inventive bioconjugated nanoparticles include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcho-line; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-laury-I ether; a surface active fatty acid, such as palmitic acid or oleic acid; fatty acids; fatty acid amides; sorbitan trioleate (Span 85) glycocholate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; and phospholipids. The lipid may be positively charged, negatively charged, or neutral. In certain embodiments, the lipid is a combination of lipids. Phospholipids useful in preparing nanocells include negatively charged phosphatidyl inositol, phosphatidyl serine, phosphatidyl glycerol, phosphatic acid, diphosphatidyl glycerol, poly(ethylene glycol)-phosphatidyl ethanolamine, dimyristoylphosphatidyl glycerol, dioleoylphosphatidyl glycerol, dilauryloylphosphatidyl glycerol, dipalmitotylphosphatidyl glycerol, di stearyloylphosphatidyl glycerol, dimyristoyl phosphatic acid, dipalmitoyl phosphatic acid, dimyristoyl phosphitadyl serine, dipalmitoyl phosphatidyl serine, phosphatidyl serine, and mixtures thereof. Useful zwitterionic phospholipids include phosphatidyl choline, phosphatidyl ethanolamine, sphingomyeline, lecithin, lysolecithin, lysophatidylethanolamine, cerebrosides, dimyristoylphosphatidyl choline, dipalmitotylphosphatidyl choline, di stearyloylphosphatidyl choline, dielaidoylphosphatidyl choline, dioleoylphosphatidyl choline, dilauryloylphosphatidyl choline, 1-myristoyl-2-palmitoyl phosphatidyl choline, 1-palmitoyl-2-myristoyl phosphatidyl choline, 1-palmitoyl-phosphatidyl choline, 1-stearoyl-2-palmitoyl phosphatidyl choline, dimyristoyl phosphatidyl ethanolamine, dipalmitoyl phosphatidyl ethanolamine, brain sphingomyelin, dipalmitoyl sphingomyelin, distearoyl sphingomyelin, and mixtures thereof. Zwitterionic phospholipids constitute any phospholipid with ionizable groups where the net charge is zero.

### Detecting Quantum Dot Binding

In one aspect, the invention includes detecting the binding of a quantum dot to a target. In one embodiment, the IL13QD binding to an IL13-specific receptor in a biological sample is detected. In one embodiment, the IL13-specific receptor is IL13Rα2. In another embodiment, the biological sample is serum, CSF, urine or other biological fluids.

In another embodiment, the IL13QD binding to exosome of a biological sample is detected. In one embodiment, the level of IL13QD to an exosome binding is correlated with disease pathology or disease progression.

In one embodiment, IL13QD binding is detected using fluorescent microscopy, immunohistochemistry, flow cytometry, agarose gel electrophoresis, immunoassay, atomic force microscopy or a combination thereof.

### Kits

The present invention also pertains to kits useful in the methods of the invention. Such kits comprise various combinations of components useful in any of the methods described elsewhere herein, including for example, IL13QD or materials for producing IL13QD, materials for quantitatively or qualitatively analyzing a the binding of IL13QD to a target in a sample, and instructional material. For example, in one embodiment, the kit comprises components useful for the quantification of the quantum dot of the invention to a target in a biological sample. In another embodiment, the kit comprises components useful for the quantification of IL13QD binding to an IL13-specific receptor or exosome in biological sample.

In a further embodiment, the kit comprises the components of an assay for monitoring the effectiveness of a treatment administered to a subject in need thereof, containing instructional material and the components for determining whether the level IL13QD binding in a biological sample obtained from the subject is modulated during or after administration of the treatment. In various embodiments, to determine whether the level of a biomarker of the invention is modulated in a biological sample obtained from the subject, the level of IL13QD binding is compared with the level of at least one comparator control contained in the kit, such as a positive control, a negative control, a historical control, a historical norm, or the level of another reference molecule in the biological sample. In certain embodiments, the ratio of the IL13QD binding to a target and to a reference molecule is determined to aid in the monitoring of the treatment.

In certain embodiments, the kit comprises a means for making IL13QD. In further aspects, the means for making IL13QD comprises reagents necessary to make IL13QD.

In certain embodiments, the kit comprises a means for enriching or isolating exosomes in a biological sample. In further aspects, the means for enriching or isolating exosomes comprises reagents necessary to enrich or isolate exosomes from a biological sample. In some embodiments, the kit comprises a means for enriching CSF exosomes.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLE 1

### Interleukin-13 Conjugated Quantum Dots for Identification of Glioma Stem Cells and their Extracellular Vesicles

Experiments were designed to develop a method to detect glioma tumor associated stem cells and the exosomes which can facilitate an early diagnosis of glioblastoma recurrence, and may distinguish them from pseudoprogression (Jahangiri and Aghi, 2012 Neurosurg Clin N Am 23:277-87; Van Mieghem et al., 2013, Eur J Neurol 20:1335-41) and could provide the first practical screening test for the populations at increased risk of developing brain tumors (Ostrom et al., 2014, Neuro Oncol 16:896-913).

The materials and methods employed in this example are now described.

Materials

Cadmium selenide (CdSe) quantum dots surface modified with polyethylene glycol (PEG) and carboxylic acid functional groups used for these studies were purchased from Ocean Nanotechnology, Inc. 1-Ethyl-3-(3-dimethylaminopropyl) carbodiimide (EDC), a water soluble carbodiimide was purchased from Thermoscientific, Inc. Sephadex G25M columns were purchased from GE Healthcare, Inc. YM-30 centrifugal concentrators were from Amicon, Inc. Fluorescent dye PKH67 was purchased from Sigma Aldrich, Inc. Human glioma cells U251 was obtained from American type culture collection (ATCC) and the human glioma stem cells T3691 and T387 were provided by Dr. Jeremy Rich, Cleveland Clinic Lerner Research Institute. Human IL13 protein (recombinant) was expressed in E. Coli and purified as described by us previously (Madhankumar et al., 2006, Mol Cancer Ther 5:3162-9).

Preparation and Characterization of IL13 Conjugated Quantum Dots

The quantum dot was conjugated to IL13 or BSA protein by EDC chemistry using EDC as conjugating agent (Tomlinson et al., 2005, Methods Mol Biol 303:51-60; Gupta et al., 2007, J Pharm Pharmacol 59:935-40). Briefly, the carboxylated quantum dots at 2 μM concentration were reacted with 10 equivalents of IL13 protein in the presence of EDC for 1 hour at room temperature under mild agitation. The excess unreacted EDC in the reaction mixture was removed by passage through a Sephadex G25M column. The eluted fractions from the column were concentrated in an YM-30 centriprep concentrator. The conjugation of the nanoparticles was confirmed by agarose gel electrophoresis and dot blots. The particle size before and after conjugation was also verified using a zetasizer (Malvern Nano ZS).

Surface Morphology

The images indicative of the surface morphology of quantum dots were obtained using a Multimode Atomic Force Microscopy (AFM) with a Nanoscope IIIc control system (software version 5.12r3, Digital Instruments, Santa Barbara, Calif.) operating under tapping mode (intermittent contact). The image acquisition was performed under ambient conditions.

Exosomes Isolation and Purification

Exosomes were isolated from the glioma stem cells (designated as T3691 and T387) and from the CSF of the brain tumor patients. The exosomes were isolated by differential centrifugation followed by ultracentrifugation method as previously described (Kesimer et al., 2009, FASEB J 23:1858-68; Wang et al., 2007, Xi Bao Yu Fen Zi Mian Yi Xue Za Zhi 23:1119-21).

Briefly, for isolating the exosomes from the cells, the cells were cultured to 80% confluence in glioma stem cell culture condition medium. As the stem cell culture medium does not contain serum in its composition, the medium was used as it is for culturing the cells without need to separate the exosomes that might be present in the serum. The condition media was collected 72 h from the time of starting the culture and subjected to differential centrifugation with the following sequential spins: 300 g for 10 minutes, 16000 g for 20 minutes, filtration through 0.22 μm membrane filtration and finally 100,000 g for 2 hours at 4° C. For isolating the exosomes from the CSF similar method as detailed above was followed after diluting the CSF with equal volume of phosphate buffered saline prior to centrifugation steps. The pelleted exosomes were re-suspended in sterile phosphate buffered saline.

Slot Blot/Dot Blot Characterization of Exosomes

Slot blot was performed by blotting 50 μl of 0.5 mg/mL concentration of exosomes per well in a slot blot apparatus and transferred to a nitrocellulose membrane. The membrane was probed with IL13Rα2 antibody (mAb) and CD63 rabbit mAb for determining their expression level in exosomes, followed by respective secondary antibody (HRP conjugate) and chemiluminescent substrate treatment, before developing the blot.

Acetyl Choline Esterase (AChE) Activity Characterization of Exosomes

To 25 μl of exosomes 100 μl of 12.5 mM acetyl choline (M.W. 181.66) and 100 μl of 1 mM 5,5-dithiobis(2-nitrobenzoic acid) (M.W. 396.35) were added and adjusted to a final volume of 1 mL. The resulting contents were transferred immediately to a black walled 96 well plate (200 μl per well) and incubated at 37° C. for 10, 20 and 30 minutes. At the end of the incubation time the absorbance at 412 nm was recorded using a plate reader as a measure of AChE esterase activity. The values are plotted as arbitrary values for each of the exosomes.

Binding of IL13QD to Glioma Cells

In vitro binding experiments were performed using IL13QD and unconjugated quantum dots on U251 human glioma cells and CD133 positive glioma stem cells (designated T3691). To demonstrate the expression of IL13Rα2 receptor in T3691 GSC and in U251 glioma cells, the cells were cultured in monolayer using geltrex matrix. T3691 cells were also cultured as aggregated neurospheres and were subjected to immunocytochemistry after treating the cells with antibody for IL13Rα2 and subsequent treatment with Alexa Fluor 488 conjugated secondary antibody. For the binding studies 20,000 cells per well were plated in a chamber slide which was cultured at 37° C. These cells were exposed to 100 fold molar excess of human recombinant IL13 protein with respect to the quantum dot concentration for 1 h. These cells were then exposed to quantum dots at 20 nM at 4° C. for 10 minutes in the case of U251 and 30 minutes in the case of T3691 cells. Subsequently the excess quantum dots with media from the cells were removed and replaced with fresh media and incubated at 37° C. for 1 h and the cells were fixed with 4% paraformaldehyde. After DAPI staining the slides were gel mounted and dried and the images were captured in a fluorescent microscope (Nikon Eclipse 80i).

Force Map Investigation

IL13QD was conjugated to the AFM probe using an established method (Xu and Siedlecki, 2009, Langmuir 25:3675-81). To determine the functional integrity of the IL13QD, AFM was used to acquire the interaction force between the quantum dots and the glioma cells cultured on the coverslips or T3691 exosomes bonded to the coverslip.

AFM Force Measurement in the Presence of IL13QD

IL13 conjugated quantum dots were covalently coupled to AFM probes using functionalized micro glass bead (size ˜4 μm in diameter) which was attached on Si₃N₄ triangular cantilevers (Veeco Instruments, Santa Barbabra, Calif.), as described elsewhere (Xu and Siedlecki, 2009, Langmuir 25:3675-81). Before performing the conjugation, the glass beads on the probes were functionally modified to bear amino groups by treating them in a 1% (v/v) solution (in ethanol) of (3-aminopropyl)triethoxysilane (Gelest, Inc., PA) for 1 h at room temperature. The probes were then extensively washed with Millipore water and treated with 10% glutaraldehyde for 1 h at room temperature. The probes were then again washed with Millipore water to remove the non-specifically bound glutaraldehyde. The activated probe was then incubated in IL13 conjugated quantum dots at a concentration of 20 nM for 1 h. It has been previously reported that this method of attachment of protein provides sufficient flexibility for the protein orientation for proper binding (Chowdhury and Luckham, 1998, Colloids and Surfaces A: Physicochemical and Engineering Aspects 143:53-7).

Interaction of IL13QD Modified AFM Probe with Glioma Cells

To determine the functional integrity of the IL13 protein conjugated to quantum dots, a Multimode AFM with a Nanoscope Ma control system (software version 5.2r3, Digital Instruments, Santa Barbra, Calif.) operating in force volume image mode was used to acquire the interaction forces between the IL13QD and the glioma cells. Briefly, U251 glioma cells were cultured on a coverslip in complete medium for 24 hrs, and then rinsed with buffer and mounted onto AFM stage. Force curves were collected at 16×16 pixels in force volume image mode over an area of 20×20 μm² at scan rate of 1 Hz. The trigger mode was set at a relative deflection threshold of 50 nm so that the total loading force was ˜3.0 nN. In order to study the effect of contact time on interaction forces, the forces were measured at different delay time (0 s and 1 s). The spring constants of cantilevers were determined using thermal tuning method. Force curve data were extracted from AFM files and analyzed off-line with tools developed using Matlab software (version 6.5). The interaction force was calculated from the distance (d) between the zero deflection values to the point of maximum deflection during probe separation from surface. F=k×d, where k is the spring constant of cantilever (nN/nm), and d is the deflection distance (nm).

Interaction of IL13QD Modified AFM Probe with Exosomes

The quantum dots were conjugated to the respective proteins (IL-13 or bovine serum albumin, BSA) using EDC chemistry as described above in the conjugation sections. The glass beads on the AFM probe were pretreated with aminopropyltriethoxy silane (APTS) (1%) for 1 h at room temperature to bear amino groups. After washing thoroughly the AFM probe was treated with glutaraldehyde (10%) for 1 h at room temperature. The activated probe was then conjugated with IL13 or BSA on the quantum dots.

The exosomes were isolated from the glioma stem cell condition medium by following ultracentrifugation method or using an exosome isolation kit (Systems Biosciences Inc.) and was characterized for the presence of IL13Rα2 and other markers like CD63 and CD81. The glass coverslips were coated with biobond after soaking the coverslips in 2% acetone solution of biobond for 20 minutes. Subsequently the coverslips were washed with PBS and treated with exosome solution at a concentration of 0.5 mg/ml for 20 minutes. AFM probe was allowed to interact with the coverslip and the force curve was determined. In the control experiment, the probe modified with BSA was utilized and the force measurement was performed.

Flow Cytometry with Exosomes and Quantum Dots

To investigate the physicochemical interaction of IL13QD with exosomes, IL13Rα2 receptor expressing exosomes isolated from glioma stem cells and that obtained from the CSF of brain tumor patients were investigated for their binding affinity towards IL13QD by flow cytometry. For this study the exosomes were conjugated to the fluorescent probe PKH67 following the manufacturer's instruction. Subsequently the fluorescently labelled exosomes and IL13QD and BSA-QD at a final concentration of 20 nM were exposed to the exosomes (100 μl) suspended in phosphate buffered saline in a final volume of 1 mL and incubated at 37° C. for 2 h before subjecting them to flow cytometry.

Based on the aggregation and complex formation potential of the quantum dots in the presence of exosomes, forward scattering and sideward scattering (SSC) were measured using a flow cytometer (FACSCanto II). The percentage gated population in forward and sideward scattering was measured and compared between control and tumor targeted quantum dots.

Transmission Electron Microscopy of Exosomes after Treatment with QD

The structural integrity of exosomes was confirmed by transmission electron microscopy (TEM). Prior to TEM imaging the exosomes were fixed in formvar coated copper grid and stained with uranyl acetate. After incubation of 10 μl of the exosomes on the formvar coated copper grid, the exosomes were fixed with 4% paraformaldehyde for 20 minutes and subsequently washed with PBS and cross-linked with 1% glutaraldehyde for 5 minutes. Finally they were washed with phosphate buffered saline and stained with 1% uranyl acetate before observation by electron microscope. To determine the exosome binding potential of the IL13QD/unconjugated QD, the quantum dot (2 μl volume) was mixed with 20 μl of exosomes at a concentration of 1 mg/ml and incubated for 30 minutes prior to the coating them on grids.

The results of the experiments are now described

### Protein Conjugation and Characterization of Quantum Dots

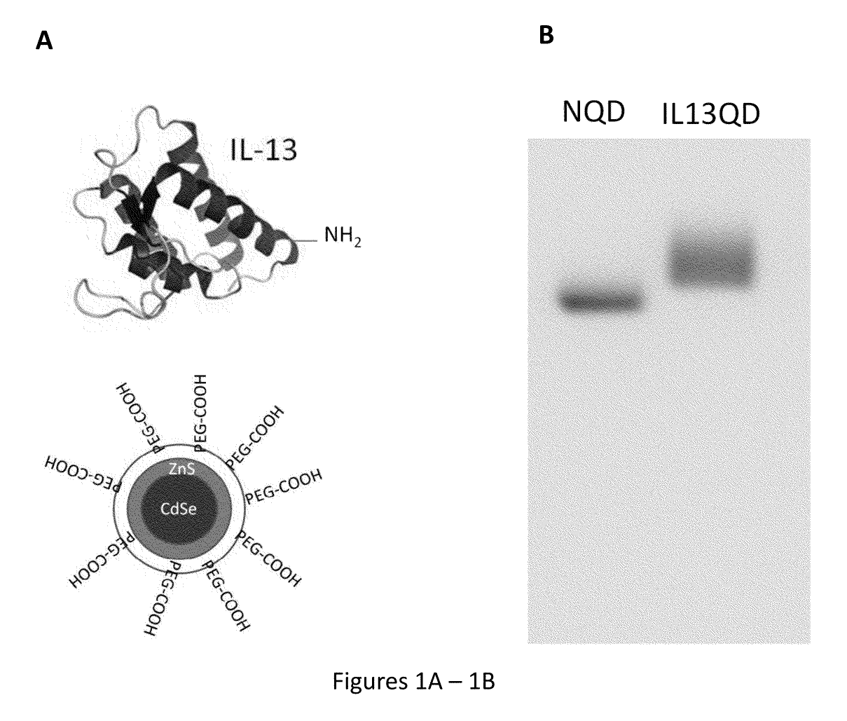

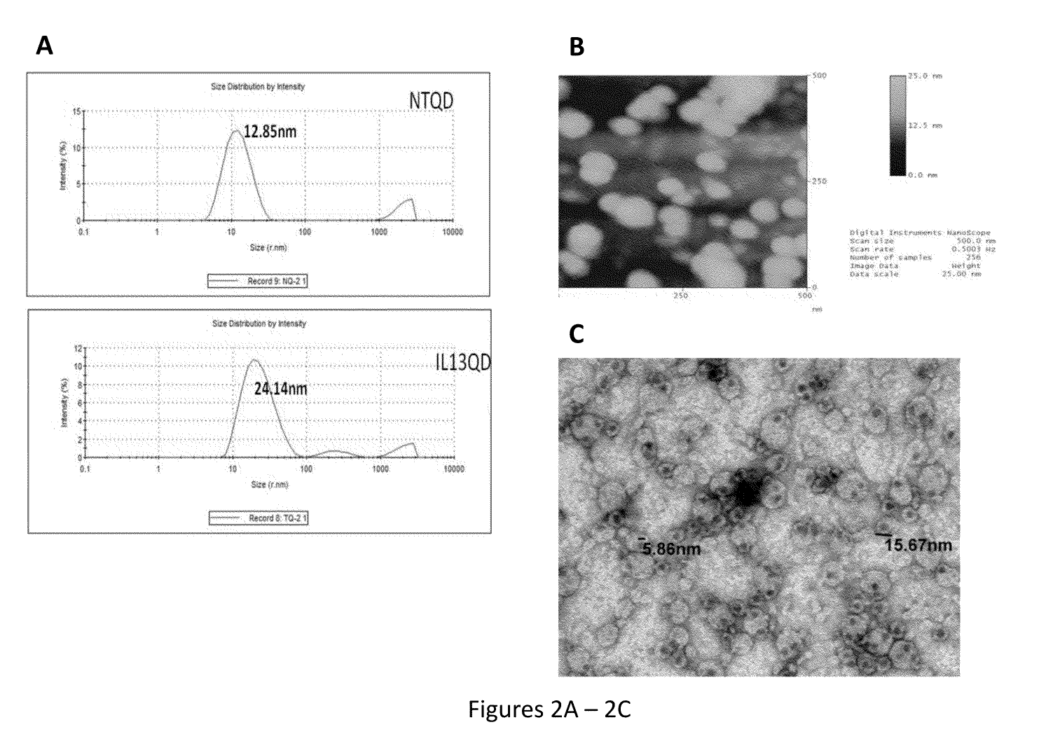

IL13 protein was conjugated effectively on the quantum dots which bears carboxyl groups on the surface of CdSe/ZnS core shell (FIG. 1A) by EDC chemistry as evidenced by gel electrophoresis (FIG. 1B). The size of the quantum dots were uniformly distributed as evidenced by particle size analyzer (zetasizer) and by atomic force microscopy (AFM) (FIGS. 2A, 2B). The mean diameter of the IL13QD was 24 nm whereas that of the non-targeted quantum dots was 13 nm. The transmission electron microscopy (TEM) images indicates a core-shell structure of the IL13 conjugated QD with approximate size around 15-20 nm (FIG. 2C).

### In Vitro Binding of IL13QD Towards Glioma Cells

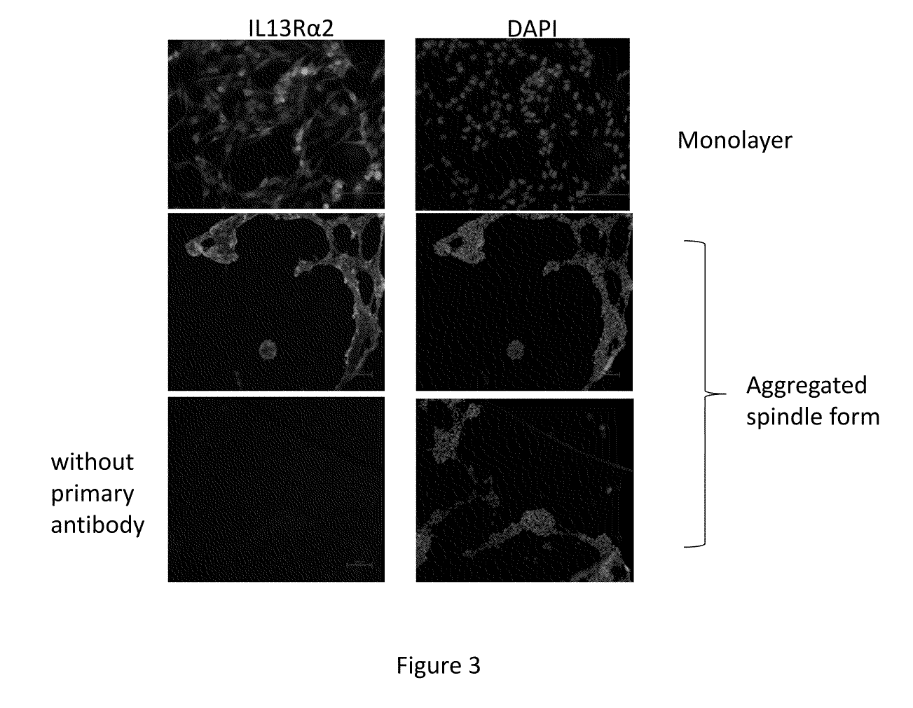

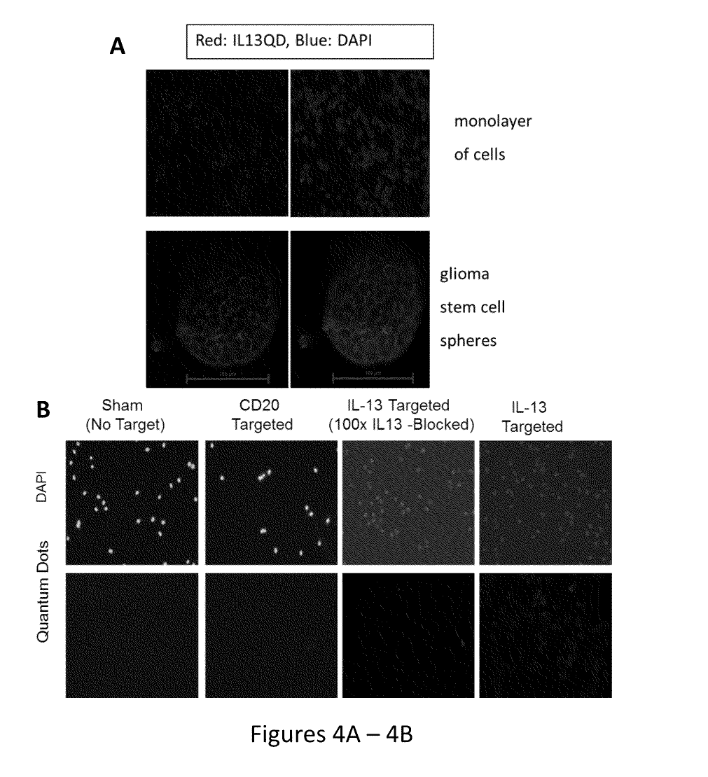

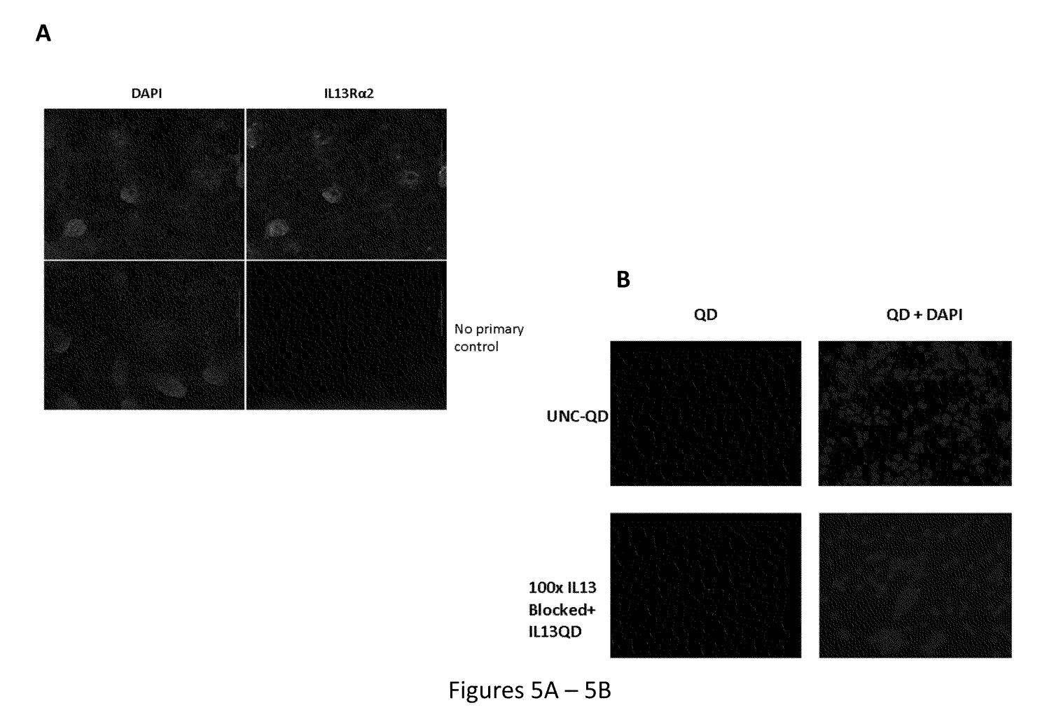

Initially, using immunohistochemistry it was demonstrated that IL13Rα2 receptor is expressed by the glioma stem cells (FIG. 3). IL13QD was able to bind effectively and selectively to the T3691 glioma stem cells as observed under fluorescence microscopy (FIG. 4A), both in monolayer culture and spheroid culture. This is also confirmed in T387 spheres, a second human CD133+ glioma stem cells (FIG. 5A). When the receptor sites on the tumor cells were blocked with 100 fold molar excess of recombinant IL13 protein, no binding of IL13QD was observed (FIG. 5B). A similar binding pattern was observed in an established glioma cell line, U251 (FIG. 4B). The binding study clearly demonstrated receptor specific binding of IL13 conjugated quantum dots to the glioma cells, whereas the sham (unconjugated) quantum dots were not able to bind. Furthermore, when conjugated to an antibody for CD20, an antigen that is specifically expressed in certain cancers like lymphoma (not expressed in GBM) no binding of the quantum dot was observed.

### The Force of Interaction Between the IL13QD and the Glioma Cells and Exosomes

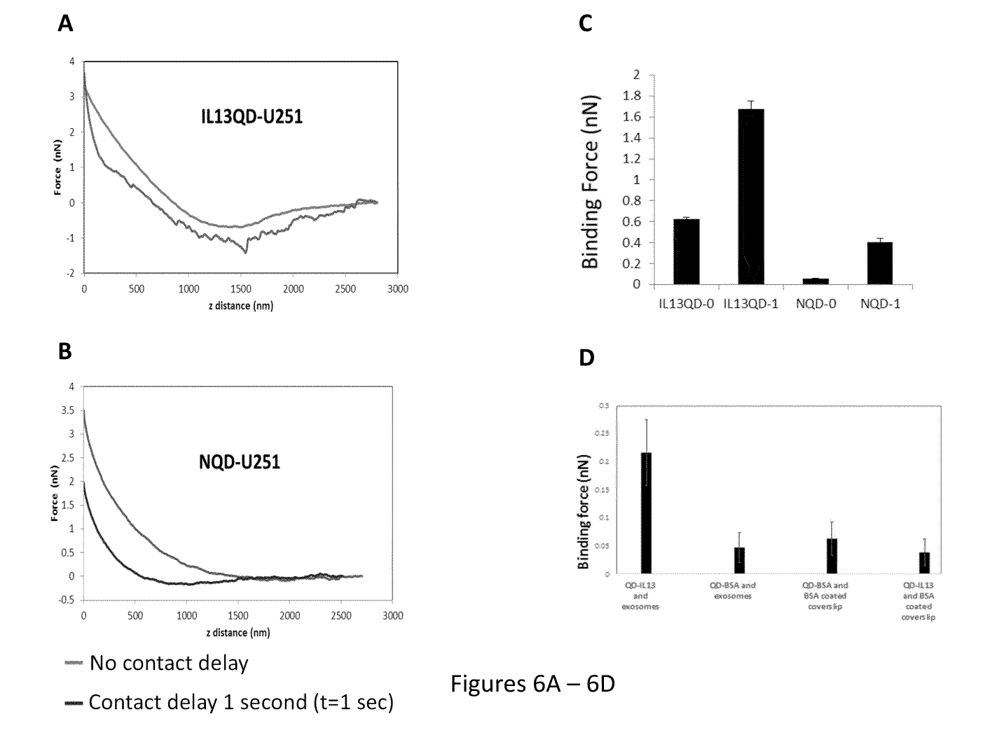

The atomic force microscopic investigation revealed higher protein-receptor interaction forces between the IL13QD and the U251 glioma cells cultured on coverslips at an exposure time of 1 second. In contrast the force curves generated in the presence of unconjugated quantum dots (NQD) were smooth without noticeable protein-receptor interaction. (FIGS. 6A, 6B). Similarly AFM study indicated a higher binding affinity of IL13QD for the T3691 secreted exosomes which were bound to the coverslip, when compared to binding affinity of BSA-QD for T3691 exosomes (FIG. 6D). Under the control condition where the biobond coated coverslips were pre-treated with BSA, minimal non-specific force of interaction was observed whereas maximum binding force was observed between the IL13QD and the exosomes (FIG. 6D). The binding force between the exosomes and the quantum dot (1-1.8 nN) was lower in magnitude (FIG. 6D) than that observed between the glioma cells and the quantum dot (0.2-0.5 nN) (FIG. 6C)

### Flow Cytometry to Determine the Binding of IL13QD to Exosomes Secreted by Cancer Cells

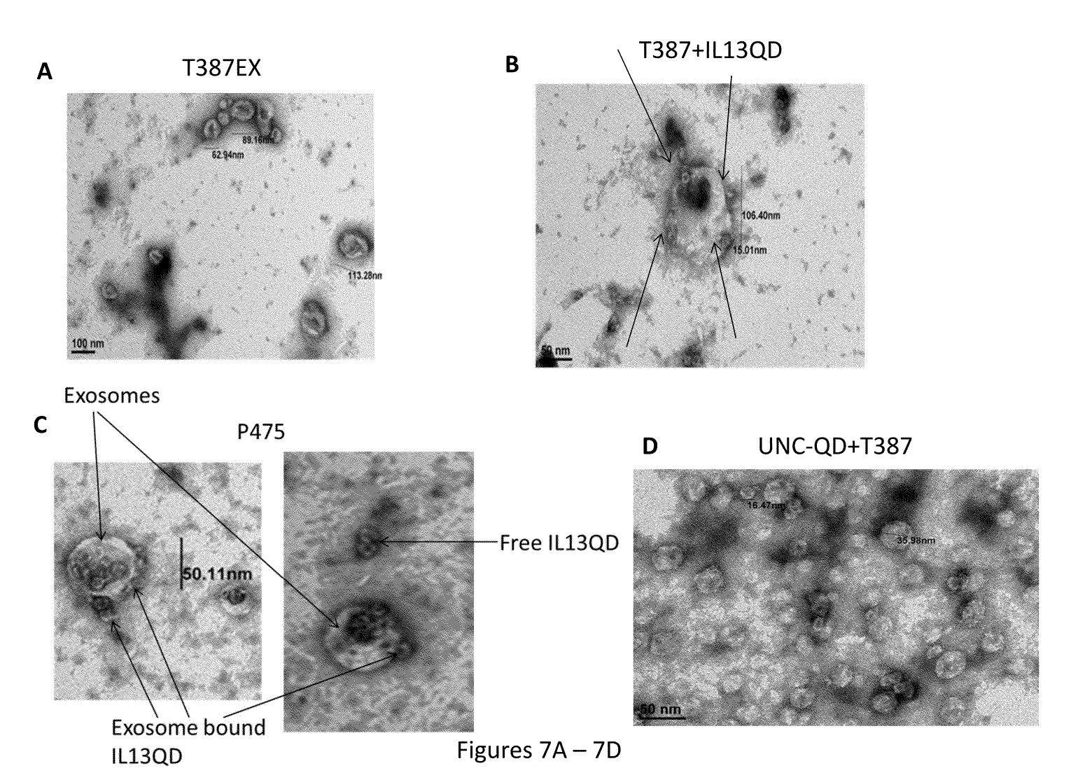

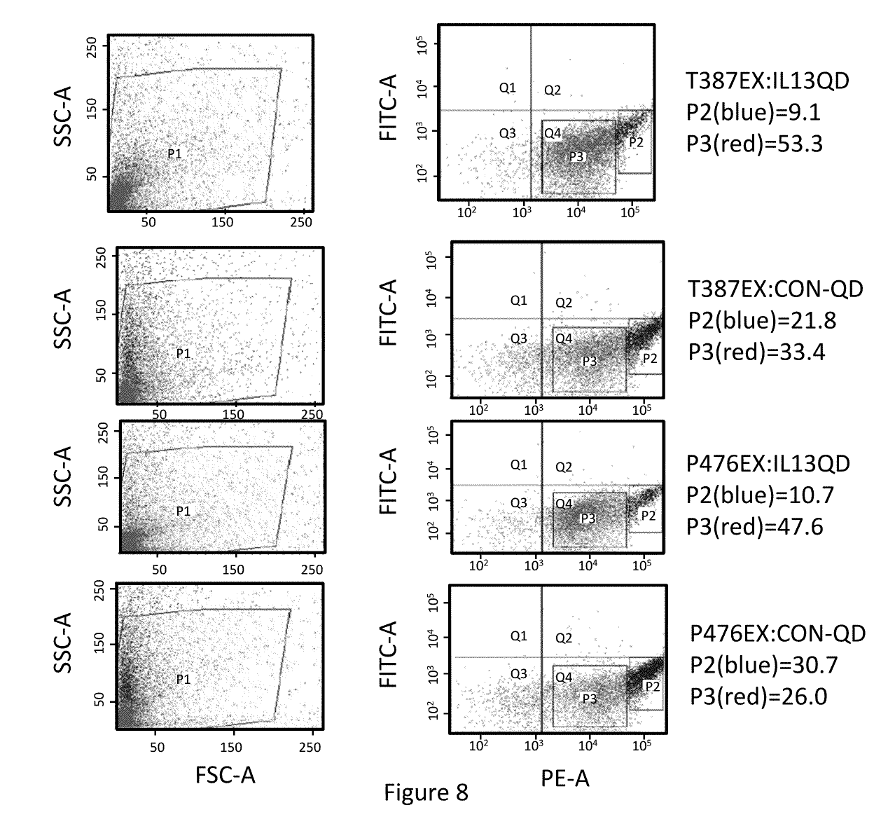

When glioma derived exosomes were incubated with IL13QD, the quantum dots tends to bind to the surface of the exosomes as evident by transmission electron microscopy images (FIG. 7). Such binding is evident both in the exosomes isolated from glioma initiating cells (T387) and from the GBM patient CSF (P475) (FIGS. 7B, 7C). The binding event between the unconjugated QD (UNC-QD) and the exosomes was observed to be minimum. This observation motivated us to perform flow cytometry to determine the nature of the binding between the exosomes and the conjugated and control quantum dots. The density plot from the flow cytometry of the quantum dot-exosome complex indicates that there is a higher sideward scattering (SSC) with the complex and smaller forward scattering (FSC) (FIG. 8). In contrast, with IL13QD a higher forward scattering and lower sideward scattering was observed when exposed to exosomes from same specimen. This result was seen with both glioma stem cell exosomes (T387-EX) and exosomes from the CSF of a patient with glioblastoma (P476) (FIG. 8). Thus in the case of T387-EX, the FSC and SSC sorted populations were 9.1% (blue) and 53.3% (red) respectively in the presence of IL13QD, whereas in the presence of control quantum dots FSC increases to 21.8% and SSC decreases to 33.4% (FIG. 8). A similar trend was observed with exosomes isolated from CSF of a patient with glioblastoma, where the FSC and SSC gated numbers were respectively 10.7% and 47.6%. In the presence of control quantum dots the FSC and SSC gated population attains a value of 30.7% and 26.0% respectively.

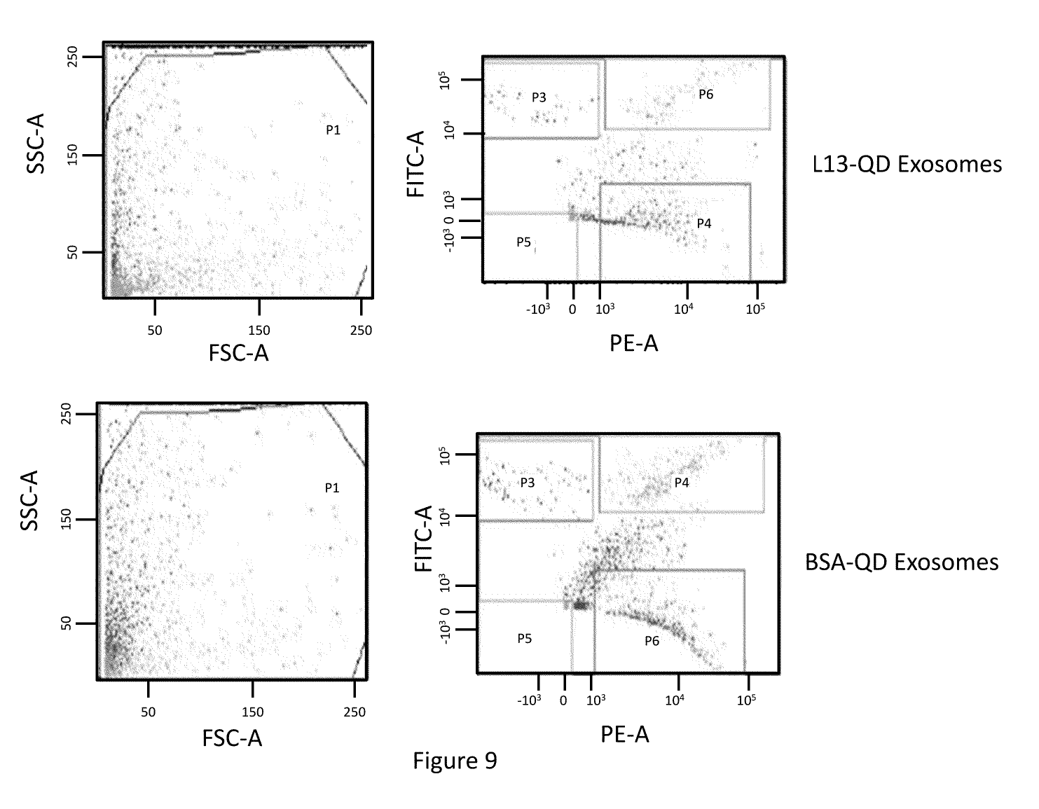

Flow cytometry performed on another glioma stem cell exosomes (T3691 EXO) using IL13 conjugated quantum dots and BSA conjugated quantum dots indicated similar observation. As evident from FIG. 9, forward scattering is much higher in IL13QD (60% of total event) compared to that of BSA-QD (22% of total event). In addition the green fluorescence from the exosomes were observed to be decreased in the IL13QD (P6=3.4% total event) compared with BSA-QD (P6=9.1%).

### Detection of IL13Rα2 and Exosomes in the Serum or CSF Utilizing the IL13QD

IL13Rα2 is one possible marker for reliable, early detection of new or recurrent cancer from easily accessible biologic fluids such as blood, urine or cerebrospinal fluid. IL13Rα2 is expressed in several malignant tumors, including adenocarcinoma, pancreatic cancers and GBM and is strongly correlated with malignancy and invasiveness (Jain et al., 2012, Cancer 118:5698-708; Fujisawa et al., 2009, Cancer Res 69:8678-85; Debinski et al., 1999, Clin Cancer Res 5:985-90).

A compelling reason to attempt to utilize quantum dots to detect IL13Rα2 is their optimal size distribution 1-25 nm and their distinct fluorescence properties, both of which allow us to monitor their dynamics in the membrane and in solution form. The size of the quantum dots increases upon conjugation with IL13 protein from 12 nm to 24 nm. The surface of the quantum dots were decorated with polyethylene glycol groups for enhanced circulation half-life in biological systems (FIG. 1; Leung, 2004, Quantum dot800-poly(ethylene glycol)-c(Arg-Gly-Asp-d-Tyr-Lys). Although these semiconductor based quantum dots, contain carboxyl groups on the surface, these are neutralized upon conjugation and the conjugated quantum dots with neutral charge have been reported previously to possess negligible non-specific interaction with cell membranes (Park et al., 2011, Advanced Functional Materials 21:1558-66). This is evident from studies where the IL13QD demonstrate strong binding affinity to the glioma cells and stem cells expressing the oncogenic IL13Rα2 receptor, but negligible binding of the unconjugated quantum dots to the glioma cells (FIG. 5B). The expression of IL13Rα2 in the patient derived glioma stem cells (T3691 and T387, FIGS. 3, 5A) clearly signifies the potential to utilize IL to detect them in vitro. The binding phenomenon of IL to the glioma stem cells in both monolayer and spheroid form further indicates the potential of the IL13QD to detect the stem cells when present in isolated form or as tumor associated spheres. Specificity for the IL13Rα2 was shown by blocking uptake with 100 fold excess of IL13 protein. The second line of evidence for selective uptake via IL13Rα2 arises from the failure of CD20-MAb conjugated quantum dots to bind to the glioma cells. CD20 is a B-lymphocyte antigen predominantly activated in lymphoma but not in other cancers and was used as a negative control. (FIG. 4B)

AFM study indicates the functional significance of the magnitude of force between the IL13QD and the cancer cells and tumor associated exosomes. Conjugation of IL13 protein on the surface of the quantum dots does not compromise its ability to bind towards glioma cells. In terms of magnitude the binding force between the IL13QD and the U251 glioma cell is at least 4 fold higher than that between unconjugated quantum dots and U251 cells (FIGS. 6A, 6B). Similarly the binding force between the glioma stem cell secreted exosomes and IL13QD is higher in magnitude than a non-specific quantum dots (FIG. 6C). This approach would be clearly important in terms of identifying and differentiating the exosomes based on the magnitude of force of interaction and subsequently to correlate with patients diagnosis, which is a future step towards developing a diagnostic tool.

Flow cytometry study indicates the propensity of the IL13QD to bind to the tumorigenic receptor protein expressed on the exosomes that are specifically secreted by glioma stem cells and the exosomes that are found in the CSF. The density plot from the flow cytometry also reveals a decrease in the sideward scattering of the QD-exosome complex. Since sideward scattering generally correlates with the complexity of the particles or subject under study, these data suggest that both glioma stem cell exosomes and patient CSF derived exosomes form a physical complex with control quantum dots resulting in higher sideward scattering in both glioma stem cell exosomes and patient CSF derived exosomes (21.8% and 30.6% respectively). However in the presence of IL13QD the heterogeneity of the complex is reduced, presumably due to receptor specific binding of the QD on the surface of the exosomes leading to minimization of sideward scattering (9.1 and 10.7% with T387-EX and P476-EX respectively). This observation indicates that the exosome-quantum dot binding is potentially receptor mediated. A second investigation on glioma stem cell exosomes (T3691EX) and IL13/BSA conjugated quantum dots supports out initial observation. A decrease in the sideward scattering and increase in the forward scattering with IL13QD compared to non-specific BSA conjugated quantum dots (FIG. 9) probably due to increase in the size of the resulting complex with IL13QD. As the exosomes are fluorescently labeled a decrease in the fluorescence gated population was observed (from 9.1% to 3.4%) indicating the possibility that binding event of IL13QD on the exosomes might decrease the FITC intensity. As the receptor IL13Rα2 is correlated with malignancy, forward and sideward scattered particle population along with FITC sorted numbers, followed by their decrease upon binding with IL13QD might indicate expression level of IL13Rα2 on the exosomes which in turn may correlate with the disease pathology.

IL13Rα2 receptor expressing exosomes plays a significant role in the tumorigenic property. Detecting the presence of exosomes in the serum or CSF utilizing the IL13QD would lead to the development of a simple diagnostic test to detect early tumor recurrence, to distinguish recurrent tumor from pseudoprogression, and perhaps to diagnose patients in high-risk groups.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of detecting an IL13-specific receptor in a sample, the method comprising:
contacting a portion of a sample with an IL13 conjugated quantum dot (IL13QD) under conditions suitable for binding of the IL13QD dot with an IL13-specific receptor and detecting the IL13-specific receptor.

2. The method of claim 1, wherein the detecting step comprises one or more of fluorescence microscopy, flow cytometry, immunoassay, atomic force microscopy, and agarose gel electrophoresis.

3. The method of claim 1, wherein the quantum dot comprises a material selected from the group consisting of cadmium selenide, zinc sulfur, silicon and any combination thereof.

4. The method of claim 1, wherein the quantum dot is surface modified with at least one selected from the group consisting of polyethylene glycol (PEG) and a carboxylic acid functional group.

5. The method of claim 1, wherein the sample comprises one or more of serum and cerebrospinal fluid.

6. The method of claim 1, wherein the IL13-specific receptor is IL13Rα2.

7. A method of detecting an exosome in a sample comprising:
contacting a portion of a sample with an IL13 conjugated quantum dot under conditions suitable for binding of the IL13 conjugated quantum dot with an exosome and detecting the exosome.

8. The method of claim 7, wherein the exosome is a tumor associated exosome.

9. The method of claim 7, wherein the quantum dot is surface modified with at least one selected from the group consisting of polyethylene glycol (PEG) and a carboxylic acid functional group.

10. The method of claim 7, wherein the exosome expresses an IL13-specific receptor.

11. The method of claim 10, wherein the IL13-specific receptor is IL13Rα2.

12. A method for diagnosing cancer in a subject, the method comprising:
determining the level of IL13 conjugated quantum dot (IL13QD) binding to a target;
comparing the level of the IL13QD binding to the target in the biological sample of the subject with a comparator control; and
diagnosing the subject with cancer when the level of the IL binding to the biological sample of the subject is altered at a statistically significant amount when compared with the level of IL13QD binding to the comparator control.

13. The method of claim 12, wherein the step determining the level of IL13QD biding to a target further comprises
obtaining a biological sample of the subject;
contacting a portion of the biological sample with a IL13QD; and
determining the level of IL13QD binding to the target in the biological sample.

14. The method of claim 12, wherein the step determining the level of IL13QD biding to a target further comprises
administering to the subject a composition comprising the IL13QD; and
determining the level of IL13QD binding to the target in situ.

15. The method of claim 12, wherein the biological sample is selected from the group consisting of serum, cerebrospinal fluid, urine and an exosome isolated from cerebrospinal fluid.

16. The method of claim 12, wherein the determining the level of IL13QD binding step comprises one or more of fluorescence microscopy, flow cytometry, immunoassay, atomic force microscopy, and agarose gel electrophoresis.

17. The method of claim 12, wherein the level of the IL13QD binding to the biological sample of the subject is increased at a statistically significant amount when compared with the level of IL13QD binding to the comparator control.

18. The method of claim 12, wherein the cancer is selected from the group consisting of adenocarcinoma, pancreatic cancers sarcoma, peripheral nerve tumors, and glioblastoma.

19. The method of claim 12, wherein the quantum dot comprises a material selected from the group consisting of cadmium selenide, zinc sulfur, and any combination thereof.

20. A conjugated quantum dot, wherein the conjugated quantum dot binds to an IL13-specific receptor.

21. The conjugated quantum dot of claim 20, wherein the conjugated quantum dot comprises IL13.

22. A kit for detecting an IL13-specific receptor, the kit comprising an IL13 conjugated quantum dot (IL13QD).
